(19) United States
(12) Patent Application Publication
(10) Pub. No.: US 2005/0228696 A1
(43) Pub. Date: Oct. 13, 2005

(54) Method of making appointments for medical examinations for patients in medical facilities and appointment-making system

(76) Inventor: Hiroshi Egawa, Tokyo (JP)

Correspondence Address: BRINKS HOFER GILSON & LIONE, P.O. BOX 10395, CHICAGO, IL, 60610, US

(21) Appl. No.: 11/048,993

(22) Filed: Feb. 2, 2005

Related U.S. Application Data
(63) Continuation-in-part of application No. PCT/JP02/08138, filed on Aug. 8, 2002.

(51) Int. Cl. G06F 17/60

(52) U.S. Cl. 705/2

(57) ABSTRACT

The present invention is directed to a method of making a medical examination appointment and a system that implements such a method. In such a system, performance data is stored for each instance of examination. Based on the stored performance data, the number of appointments acceptable for each time frame is updated. When an appointment request designating at least one kind of examination is received, one or more time frames is determined which are available to accept the appointment request. In determining one or more time frames, (a) it is also determined if a physician needs to attend the examination, (b) it is also determined if the examinations can be conducted on the same day if more than one kind of examination is designated, and (c) priority among the examinations is determined if more than one kind of examination is designated. Then, the available one or more time frames are presented for selection.

This is a continuation-in-part of PCT/JP02/08138 filed on Aug. 8, 2002, the entire content of which is hereby incorporated by reference.

### TECHNICAL FIELD

The present invention relates to a method of making appointments for medical examinations and the like for patients in hospitals or other medical facilities and an appointment-making system for implementing this method of making appointments.

### TECHNICAL BACKGROUND

Conventionally, known methods of making appointments for medical examinations and the like for patients in hospitals or other medical facilities include those that make use of computer systems. Known computer systems for implementing these methods of making appointments include an appointment-making system wherein: in response to a query regarding the situation of appointments for consultations and the like, a search is performed upon an appointment file that records the situation of appoints for consultations and the like, a report of the situation of pertinent appointments is made, and while referring to that situation of appointments, the scheduled date and time for an consultation or the like by the attending physician is set within a preset time frame for consultations or the like and then written to the appointment file, thus making appointments for consultations or the like (see publication of unexamined Japanese patent application (Kokai) No. JP-A-H5-225213).

Most systems that make appointments using computers, not only those used for making appointments in medical facilities, are provided with an appointment situation storage block that stores the appointment situation in a computer system, and within this appointment situation storage block is prepared a time frame in which appointments can be made. With an appointment-making system used in a medical facility, the time frame in which appointments for medical examinations or the like can be made is set up in advance with reference to rules found by experience, and prepared in the appointment situation storage block.

However, with a conventional appointment-making system used in medical facilities, it is not possible to determine the actual amount of time required for a medical examination, so it is not possible to evaluate the performance, in terms of the time required per examination, of the examination department or the like. Accordingly, once a time frame in which appointments can be made is set up, it is not possible to change the time frame to reflect the actual performance of the examination department or the like, so it has been difficult to achieve further improvements to the efficiency of work of the overall medical treatment.

Since medical examinations are intended to be done in order for more appropriate medical consultations and treatment to be performed, the setting of the examination schedule should be such that the physician treating the patient can perform the examinations when conversing with the patient at the time that the patient is being examined. However, since there are many types of medical examinations and some of the examinations cannot be performed on the same day, and there are cases in which if one examination is performed, another examination cannot be performed and the like. Regardless of this, upon reviewing the appointment-making systems actually used in medical facilities, the appointment situations for each of the various examinations are simply displayed upon a display screen, so it is extremely difficult to make appointments so as to adjust the examination schedule in consideration of the appropriate combination of examinations and sequence within the attending physician's limited consultation time. Thus, the situation is such that the appointment-making work for each examination is performed in each individual department, so after undergoing a medical consultation, the patient must make the rounds of the various examination departments and make appointments at the reception desks of each department, so this is inconvenient.

In addition, even in cases in which appointments are made from a terminal used by the attending physician, or in which appointments are made from a terminal used at the appointment reception desk of each examination department, with a conventional appointment-making system, when a query of the appointment situation is made, the result is that a calendar-format table that shows the appointment situation or namely information on appointment slots not yet filled (vacancy information) mixed together with already filled appointments is displayed upon the display screen of the person making the query. Accordingly, the attending physician or other personnel must make the appointment after searching through the information displayed in the table for vacancies, and the method of displaying these vacancies itself is one impediment to improving the efficiency of the work of making appointments.

In addition, some medical examinations require the attendance of the physician while others do not. When making appointments for medical examinations that require the attendance of the physician, it is necessary to take the schedule of the physician also into consideration, so with a conventional appointment-making system, it was not possible to determine the consultation schedule of the physician, so situations have occurred wherein, because of the examination appointment dates and times not being incorporated into the physician's consultation schedule, the examination cannot be performed on the appointed day because the physician is not present, or the patient is forced to wait for a long time until the physician finds time.

On the other hand, from the physician's perspective, rather than the date on which the examination is scheduled (date performed), the date on which the results of that examination can be obtained is a more important matter with regard to performing consultations. For this reason, when making appointments for medical examinations, while it is naturally necessary to take the date and time desired by the patient into consideration, medical examination are no more than one means to the end of performing consultations, so the date and time of the appointment should be set by the physician after determining the date when the examination results will be obtained. However, conventional appointment-making systems placed emphasis upon being able to make appointments and being able to manage appointments all at once, so no consideration was given to being able to determine when the physician will be able to obtain the examination results. In addition, the date when the examination results will be obtained is not taken into consideration at the time of making an appointment for a medical examination, so it was necessary for the patient to make a new appointment for the next consultation after the examination is complete.

The present invention was made in consideration of the aforementioned situation, and has as its object to provide a method for a physician to easily make appointments for medical examinations during consultations, an appointment-making system for implementing this method of making appointments, a computer program used in this appointment-making system and a recording medium for recording this computer program. Another object is to provide a method of making appointments whereby a physician can determine the date when examination results will be obtained and thus set up an appropriate consultation schedule, and also to provide a method of making appointments that not only improves the efficiency of appointment work but also achieves improved efficiency of examination work that is subject to appointment.

### SUMMARY OF THE INVENTION

In order to achieve the above objects the present invention provides a method of making appointments as follows:

The first aspect of the present invention is directed to an appointment-making method using a computer system to make appointments for medical examinations for patients in medical facilities. Such a method comprises the steps of: storing the appointment situation for examinations in the computer system, and extracting from the stored appointment situation for examinations one or more dates and times when appointments for specified examinations can be made.

In the above appointment-making method, limitations with respect to examinations may be stored in the computer system, and the feasibility of performing two or more specified examinations on the same day is determined in accordance with the limitations with respect to examinations thus stored. Also, the priority order of examinations may be stored in the computer system, and the sequence of two or more specified examinations is determined in accordance with the priority order of examinations thus stored.

The waiting periods until the results come back for each examination may be stored in the computer system, and by adding the waiting period until the results come back for the examination in question thus stored to the dates and times when appointments for the examinations in question can be made thus extracted, it is possible to compute the dates when the results of the examinations in question can be obtained.

The appointment situation for consultations and rules for the necessity of physician attendance for each examination may be stored in the computer system, and the necessity for physician attendance for a specified examination is determined according to the rules for the necessity of physician attendance for each examination thus stored, and in the event that the attendance of a physician is necessary, one or more dates and times when appointments for specified examinations can be made are extracted after making an inquiry into the appointment situation for consultations. Further, the appointment situation for consultations may be stored in the computer system, and from the stored appointment situation for consultations is extracted one or more dates and times when an appointment for a post-examination consultation can be made.

When appointments for two or more specified examinations are made on the same day, one or more dates and times when appointments for the examinations in question can be made may be extracted by giving priority to those dates and times that give rise to the shortest waiting times between the examinations. The examination performance of each examination may be stored in the computer system, and the time frame wherein appointments for examinations can be made is changed depending on the examination performance thus stored.

In addition, the present invention provides an appointment-making system used to make appointments for examinations for patients in medical facilities. The appointment-making system comprises an examination appointment situation storage block that stores the appointment situation for examinations, and examination appointment date and time search means for extracting, from the appointment situation stored in the examination appointment situation storage block, one or more dates and times when appointments for specified examinations can be made.

The appointment-making system may comprise a medical examination limitation storage block that stores limitations with respect to examinations, and feasibility determination means that determines the feasibility of performing two or more specified examinations on the same day in accordance with the limitations with respect to examinations stored in the examination limitation storage block. It may further comprises a priority order storage block that stores the priority order of examinations, and examination sequence determination means that determines the sequence of two or more specified examinations in accordance with the priority order of examinations thus stored.

The system may also comprise a result waiting period storage block that stores the waiting periods until the results come back for each examination, and a computation means that, by adding the waiting period until the results come back for the examination in question stored in the result waiting period storage block to the dates and times when appointments for the examinations in question can be made extracted by the examination appointment date and time search means, computes the dates when the results of the examinations in question can be obtained.

It may also comprise a consultation appointment situation storage block that stores the appointment situation for consultations and a physician attendance necessity rule storage block that stores rules for the necessity of physician attendance for each examination, wherein the examination appointment date and time search means determines the necessity for physician attendance for a specified examination according to the rules for the necessity of physician attendance for each examination stored in the physician attendance necessity rule storage block, and in the event that the attendance of a physician is necessary, further extracts one or more dates and times when appointments for specified examinations can be made after making an inquiry into the appointment situation for consultations stored in the consultation appointment situation storage block.

The system may comprise a consultation appointment situation storage block that stores the appointment situation for consultations, and a consultation appointment date and time search means that, based on the dates when the examination results will be obtained as computed by the computation means, makes an inquiry into the appointment situation for consultation stored in the consultation appointment situation storage block, and extracts one or more dates and times when an appointment for a post-examination consultation can be made. When appointments for two or more specified examinations are made on the same day, the examination appointment date and time search means extracts one or more dates and times when appointments for the examinations in question can be made by giving priority to those dates and times that give rise to the shortest waiting times between the examinations.

The system may also comprise an examination performance storage block that stores the examination performance of each examination, and appointment time frame change means that changes the time frame wherein appointments for medical examinations can be made that is prepared within the examination appointment situation storage block depending on the examination performance stored in the examination performance storage block.

The second aspect of the present invention is directed to a method of making a medical examination appointment and a system that implements such a method. In such a system, performance data is stored for each instance of examination. Based on the stored performance data, the number of appointments acceptable for each time frame is updated.

When an appointment request designating at least one kind of examination is received, one or more time frames is determined which are available to accept the appointment request. In determining one or more time frames, (a) it is also determined if a physician needs to attend the examination, (b) it is also determined if the examinations can be conducted on the same day if more than one kind of examination is designated, and (c) priority among the examinations is determined if more than one kind of examination is designated. Then, the available one or more time frames are presented for selection.

The performance data may comprise a time actually spent for an examination, the identity of a person who conducted the examination, and attributes of a patient who underwent the examination. The attributes comprise any of the age, sex and bodily condition of the patient.

The number of appointments acceptable for each time frame is updated, using the average of the number of patients actually examined over a period of time for each time frame. More specifically, the number of appointments acceptable for each time frame is updated by increasing or decreasing a number of appointments acceptable for a time frame. When updating the number of appointments acceptable for each time frame, appointments already made before the update will be left intact.

When presenting the available one or more time frames for selection, a date when results of examination become available is also presented for each of the available one or more time frames.

When determining one or more time frames available to accept the appointment request, a set of available time frames whose time intervals between the time frames are shortest is selected with priority if more than one time frame is determined necessary to fill the appointment and if more than one set of time frames are available for the appointment.

Also, along with presenting available time frames for examinations, time slots for selection which are available for post-examination consultation with a physician may be presented. The time slots are determined based on dates when examination results become available.

### BRIEF EXPLANATION OF THE DRAWINGS

FIG. 1 is a diagram showing the basic structure for implementing the method of making appointments according to the present invention;

FIG. 2 is a structural diagram of an appointment-making system according to an embodiment of the present invention;

FIG. 3 is a flowchart illustrating one example of the operation of the appointment-making system (the operation when making an appointment for a medical examination) according to this embodiment;

FIG. 4 is a flowchart illustrating one example of the operation of the appointment-making system (the operation when making an appointment for a post-examination consultation) according to this embodiment;

FIG. 5 is an example of a search result screen when making an appointment for a medical examination;

FIG. 6 is an example of a search result screen when making an appointment for a post-examination consultation;

FIG. 7 is a flowchart illustrating one example of the operation of the appointment-making system (the operation when changing the time frame in which examination appointments can be made) according to this embodiment;

FIGS. 8(A)-(E) are diagrams illustrating the processes of updating the number of appointments acceptable for time frames; and

FIGS. 9(A) and (B) are diagrams illustrating a change of a time frame by the update.

### PREFERRED EMBODIMENTS FOR IMPLEMENTING THE INVENTION

Here follows an even more detailed description of the mode of implementing the present invention made with reference to the drawings.

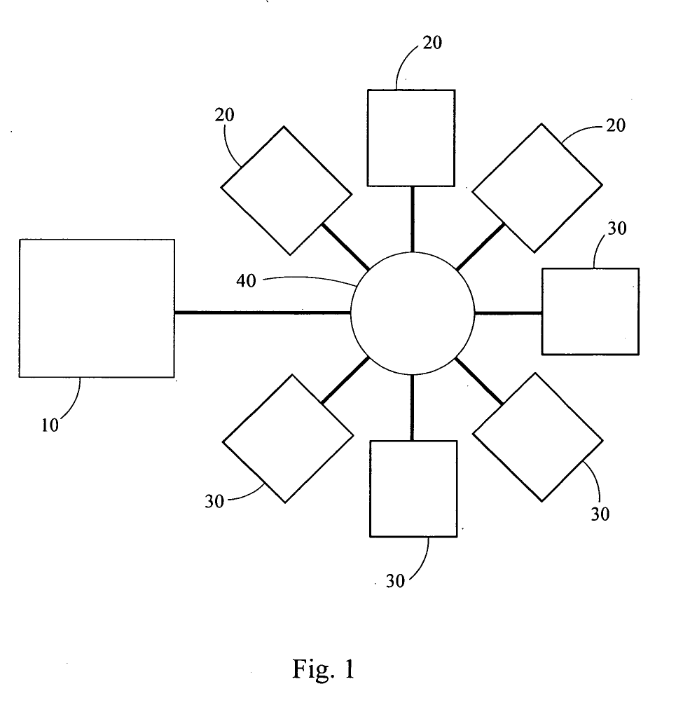

As shown in FIG. 1, the appointment-making system 10 according to the present invention which is connected via a communications network 40 to both terminals 20 that can be used by physicians and terminals 30 that are installed in various examination departments and the like, and functions as a server with respect to these terminals 20 and 30 as clients.

The communications network 40 connects the appointment-making system 10 to the physicians' and examination departments' and other terminals 20 and 30. The system 40 may be either wired or wireless as long as it is able to perform the bidirectional transmission of data.

The physicians' and examination departments' and other terminals 20 and 30 are not particularly limited as long as they are provided with communication functions whereby information can be exchanged with the appointment-making system 10, and personal computers or the like, for example, can be used for them.

Note that the physicians' terminals 20 include terminals that are usable by persons who input information to the terminals under the direction of the physicians. In addition, the terminals for various examination departments and the like 30 include not only terminals installed in the examination departments but also terminals installed at the reception desks for consultations and the like. In addition, the terminals for various examination departments and the like 30 include not only terminals installed in a single medical facility, but also terminals installed in external medical facilities that are accessed via the Internet or the like as the communications network 40.

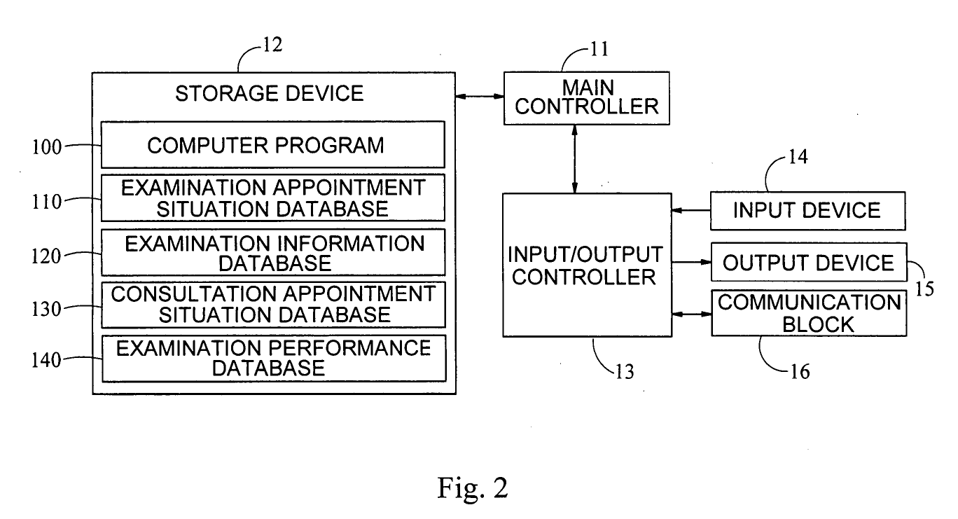

As shown in FIG. 2, the appointment-making system 10 is made up of a main controller 11 consisting of a CPU or the like, a storage device 12 consisting of semiconductor memory, a hard disk drive or the like, an input device 14 consisting of a keyboard, mouse or the like each connected via a input/output controller 13, an output device 15 consisting of a display, printer or the like, a communication block 16 that functions as a means of conducting communications with the physician's terminal 20 and the like, and other hardware.

The storage device 12 may include, as the storage media for storing various types of data, semiconductor memory and hard disks as well as floppy disks or other magnetic disks, CD-ROM, DVD or other optical disks, MO, MD or other magneto-optical disks, for example, that are loaded into a compatible drive that is mounted in the appointment-making system, or other storage media.

Upon the storage device 12 is installed a stipulated OS as well as a computer program 100 that functions in this embodiment as control means for implementing the method of making appointments according to the present invention.

The computer program 100 is made up of an examination appointment date and time search means, feasibility determination means, examination sequence determination means, computation means, consultation appointment date and time search means, appointment-making time frame changing means, information sending means, appointment date and time setting means, and the like.

The examination appointment date and time search means extracts, from the appointment situation for examinations that is recorded in an examination appointment situation storage block (to be described later), one or more dates and times on which a specified appointment for an examination may be set. In addition, the examination appointment date and time search means uses rules for the necessity of physician attendance for each individual examination stored in the attendance necessity storage block (to be described later) to determine the necessity of physician attendance for a specified examination, and if attendance by the physician is necessary, it further examines the appointment situation for consultations recorded in a consultation appointment situation storage block (to be described later) and then extracts one or more dates and times on which an appointment for the examination in question may be made.

The feasibility determination means determines the feasibility of performing two or more specified examinations on the same day in accordance with limitations with respect to examinations that are stored in an examination limitation storage block (to be described later).

The examination sequence determination means determines the sequence of two or more specified examinations in accordance with a priority order for examinations stored in a priority order storage block (to be described later).

The computation means computes the date on which the results of the examination in question can be obtained by adding a waiting period until the results of the examination in question come back that is stored in a result waiting period storage block (to be described later) to the aforementioned dates and times on which an appointment for the examination in question may be made that are extracted by the examination appointment date and time search means.

Based on the date, computed by the aforementioned computation means, on which the examination results can be obtained, the consultation appointment date and time search means extracts, from the appointment situation for consultations that is recorded in the consultation appointment situation storage block (to be described later), one or more dates and times on which a specified appointment for a consultation may be made.

The appointment-making time frame changing means, depending on the examination performance stored in an examination performance storage block (to be described later), changes the time frame in which examination appointments can be made that is prepared within the examination appointment situation storage block (to be described later).

The information sending means sends various types of data to the physician's terminals and the like.

The appointment date and time setting means sets dates and times that are available for examinations as selected by a physician or others and received from the physician's terminal or other terminals in a time frame available for the making of appointments for medical examination that is prepared within the examination appointment situation storage block (to be described later), and storing it in the examination appointment situation storage block.

Here follows a description of the operation of an appointment-making system 10 that is controlled by a computer program 100 consisting of each of the means described above.

Note that the computer program 100 can be stored and distributed on computer-readable storage media, e.g. floppy disks or other magnetic disks, CD-ROM, DVD or other optical disks, MO, MD or other magneto-optical disks, for example, or may be distributed via a communications network without the interposition of magnetic media.

Within the storage device 12 are also stored various types of databases that function as the storage blocks for storing the examination appointment situation, limitations with respect to examinations, priority order for examinations, waiting period until the results of the examination in question come back, rules for the necessity of physician attendance, consultation appointment situation, examination performance and the like.

To wit, within the storage device 12 are stored: 1) an examination appointment situation database 110 that functions as the examination appointment situation storage block that stores the examination appointment situation, 2) an examination information database 120 that functions as the examination limitation storage block that stores limitations with respect to examinations, the examination order storage block that stores the priority order for examinations, the result waiting period storage block that stores the waiting period until the results of the examination in question come back, the attendance necessity rule storage block that stores rules for the necessity of physician attendance for each examination, 3) a consultation appointment situation database 130 that functions as the consultation appointment situation storage block that stores the consultation appointment situation, and an examination performance database 140 that functions as the examination performance storage block that stores examination performance.

Within the examination appointment situation database 110 are prepared time frames in which appointments for examinations can be made. The time frames may be prepared for each examination, for each examination department, or they may be prepared for each of the examination rooms provided in each examination department, or alternately they may be prepared for each piece of examination equipment used in the examinations. In addition, these time frames may be set up based on the amount of time that it takes to perform the examination upon a single patient, or they may be set up based on the number of patients that can be examined within a fixed amount of time. To wit, depending on how these time frames are prepared, appointments for medical examinations can be made for each individual examination or they can be made for each examination department, or they can be made for each examination room provided in each examination department, or they can be made for each piece of examination equipment used for medical examination.

The “limitations with respect to examinations” stored in the examination information database 120 are defined to be rules that determine, when one is attempting to make appointments for two or more examinations on the same day, whether or not those examinations can be performed on the same day. The determination of whether or not two or more examinations can be performed on the same day is made in consideration of the nature of the individual examinations (examination methods or effects they have on the body, etc.) and the like. In addition, when appointments for two or more examinations are to be made and among them there are examinations that should be performed in priority to the other examinations, the “priority order for examinations” is defined to be the rules for the priority order for examinations so that the examinations can be performed in the correct order. In the same manner as the limitations with respect to examinations, the priority order for examinations is determined in consideration of the nature of the individual examinations and the like. In addition, the “waiting period until the results of the examination in question come back” is defined for each examination as the period from when the examination is performed until the results of that examination are known. In addition, some of the examinations require the attendance of a physician, so the “rules for the necessity of physician attendance” are defined for each examination as rules for whether or not the attendance of a physician is necessary.

Like the examination appointment situation database 110, within the consultation appointment situation database 130 are prepared time frames in which appointments for consultations can be made. These time frames may be prepared for each physician or they may be prepared for each department to which the physician belongs.

“examination performance” stored in the examination performance database 140 is defined to mean the actual time required for the examination.

Here follows a description of one example of the operation of the appointment-making system 10.

(1) Making Appointments for Medical examinations

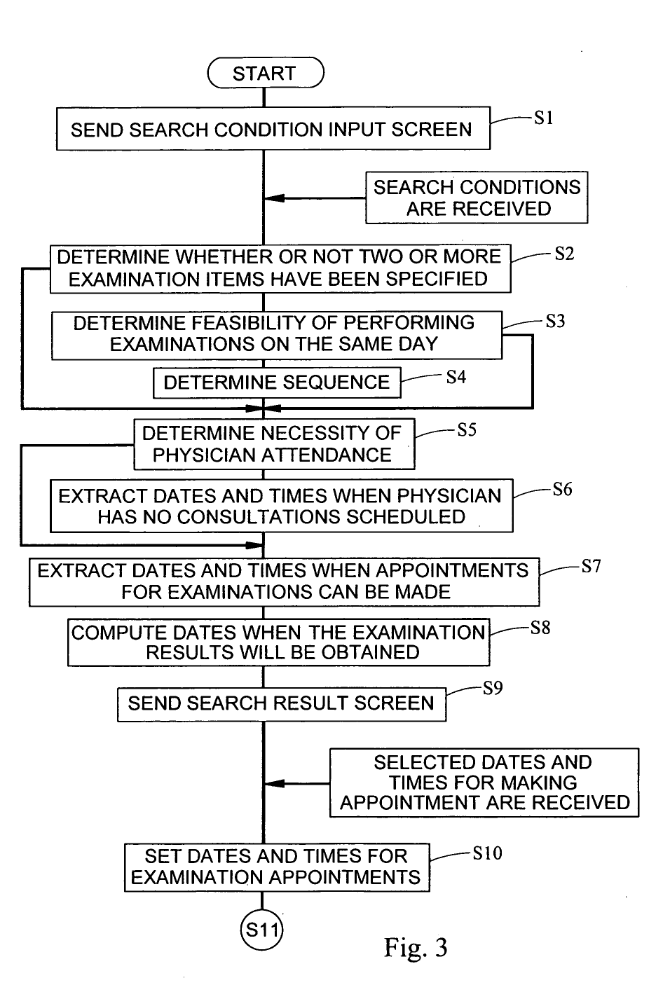

Appointments for examinations can be made using either the physician's terminal 20 or the terminals for various examination departments and the like 30, but here we shall describe the case of making an appointment using the physician's terminal 20 with reference to the flowchart shown in FIG. 3.

First, when a request to the effect that someone wishes to make an appointment is received from the physician's terminal 20, the appointment-making system 10 executes the information sending means to send a search condition input screen to the physician's terminal 20 (Step 1). Thereby, a search condition input screen is displayed upon the display of the physician's terminal 20, so based on the results of the consultation and the desires of the patient and other factors, the physician uses input fields displayed upon this screen to enter the type of medical examination (e.g., imaging examination, physiological examination, etc.), examination items (e.g., CT, MRI, X-ray, etc.), area of body (e.g., head, neck, chest, abdomen, etc.), examination method (e.g., specification of the method of scanning, etc.), desired date and time (e.g., desired date, desired day of the week, desired time, etc.) and the like, and then sends the information thus input to the appointment-making system 10.

Upon receiving the search conditions from the physician's terminal 20, the appointment-making system 10 performs the following operations. To wit, first, by executing the examination appointment date and time search means, based on the search conditions thus received, the system determines whether or not two or more examination items have been specified (Step 2). If the result of the determination is that two or more examination items have not been specified, then control advances to Step 5 (to be described later). However, if two or more examination items have been specified, next, by executing the feasibility determination means, the system accesses the examination information database 120 and, based on the limitations with respect to examinations that are stored in this database 120, determines whether or not the specified two or more examinations can be performed on the same day (Step 3). Thereby, it is possible to avoid making appointments on the same day for two examinations that cannot be performed on the same day.

If there are examinations that cannot be performed on the same day, then control advances to Step 5 (to be described later). However, if the examinations can be performed on the same day, next, by executing the examination sequence determination means, the system accesses the examination information database 120 and, based on the priority order for examinations that is stored in this database 120, determines the sequence of the two or more (Step 4). Thereby, the sequence in which the examinations are to be performed can be set appropriately. For example, if there are two examinations: “gastroscopy” and “abdominal CT” that can be performed on the same day, in Step 4, the sequence of examinations is determined such that the “abdominal CT” is done first and the “gastroscopy” is done later in order to make sure that the examination sequence is appropriate.

Once the sequence of examinations is determined, next, by executing the examination appointment date and time search means, the system accesses the examination information database 120 and, based on the rules for the necessity for physician attendance that is stored in this database 120, determines whether or not the attendance is necessary for the specified examination (Step 5). If the attendance of a physician is determined to be unnecessary as a result of this determination, then control advances to Step 7 (to be described later). However, if the attendance of a physician is determined to be necessary, by executing the examination appointment date and time search means for this examination, the system accesses the consultation appointment situation database 130 and makes a query on the appointment situation for consultations stored in this database 130, and thus extracts dates and times at which no appointments have been made for consultations with the physician who is attending the patient (Step 6). The dates and times at which no appointments have been made for consultations with the physician extracted here are added to the search conditions for extracting dates and times for which appointments for the examinations can be made. Moreover, by executing the examination appointment date and time search means based on these search conditions, the system accesses the examination appointment situation database 110 and makes a query on the appointment situation for examinations stored in this database 110, and thus extracts one or more dates and times at which appointments can be made for each of the examinations (Step 7).

With the appointment-making system 10 thus described, a determination is made as to whether or not the attendance of a physician is required for the specified examinations, and if the attendance of a physician is required, a query is made of the appointment situation for consultations and then a query of the appointment situation for examinations is made in order to extract dates and times at which the appointments for examinations can be made, so it is possible to avoid such problems as not being able to perform examinations because the physician is absent on the appointed date for the examination (date when the examination is performed).

Note that if, as a result of executing the examination appointment date and time search means, a single day on which the appointments can be made is extracted, for example examination A at 9:00 pm and examination B at 4:00 pm on the same day, but both A and B are examinations that can be performed in a few minutes then a situation occurs wherein the patient would need to spend a large part of the active time of the day as wasteful waiting time between examinations A and B in order to undergo both of these examinations. To solve this problem and prevent such situations from happening as much as possible, it is preferable for the examination appointment date and time search means to be set so that it can give priority to the extraction of appointments that give the shortest waiting times between examinations when two or more examinations are to be scheduled on the same day. Thereby, there is an advantage in that, when two or more examinations are to be scheduled on the same day, the patient can easily select those appointments that result in the shortest waiting time between examinations, and by selecting those appointments that have the shortest waiting time between examinations, the waiting time between examinations can be shortened.

Once the dates and times at which the appointments for examinations can be made are extracted, next, by executing the computation means, the system accesses the examination information database 120 and makes a query on the waiting period until the results come back for each examination stored in this database 120, reads the waiting period until the results come back for the specified examination, adds the period until the results come back for the examination in question to the dates and times for which appointments for the examinations can be made for the examination in question extracted in Step 7, and thus computes the date when the results of the examination in question can be obtained (Step 8). For example, if the examination information database 120 stores data to the effect that the waiting periods until the results of examination A and examination B come back are 5 days and 10 days, respectively, after the examination is performed, and the dates and times for which appointments for examination A and examination B can be made as extracted in Step 7 are the same day of July 15, then the dates when the results of examination A and examination B can be obtained are computed to be July 20 and July 25, respectively.

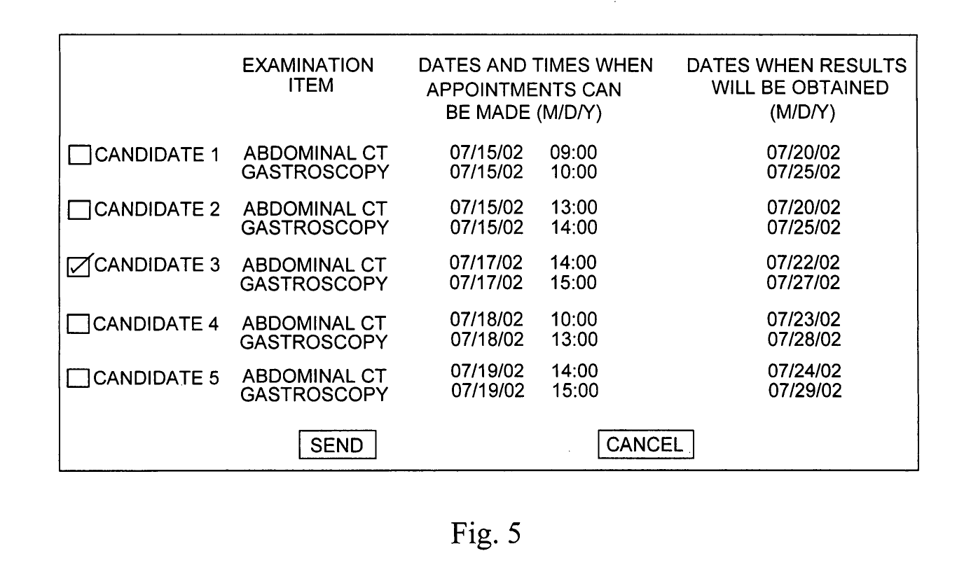

Based on Steps 7 and 8 above, once the extraction of the dates and times when the appointment for the examinations can be made and the computation of the dates when the results of the examinations in question can be obtained are performed, next, by executing the information sending means, the system sends a search result screen containing the dates and times when the appointment for the examinations can be made and the dates when the results of the examinations in question can be obtained as extracted or computed in Steps 7 and 8 to the physician's terminal 20 (Step 9). Thereby, a search result screen such as that illustrated in FIG. 5 is displayed upon the display of the physician's terminal 20.

As shown in FIG. 5, with the appointment-making system 10, rather than displaying the dates and times when appointments for examinations have been made, it is possible to display only those dates and times when appointments for examinations can be made, so there is no need to search within tables where both are displayed together for dates and times when appointments for examinations can be made, as is necessary with the prior art, and moreover, the examination items in question are displayed on a single screen, so there is no need to open individual screens for each of the examination items, as is necessary with the prior art. Accordingly, appointments can be made quickly and efficiently without putting an excessive burden on physicians or others. In addition, the dates when the examination results will be obtained are also displayed for each set of dates and times when the appointments for examinations can be made, so the physician or other personnel is able to select the dates and times when the appointments for examinations can be made based on the dates when the examination results will be obtained.

The physician may ask the patient for an opinion and add this to the physician's own judgment and then check one of the checkboxes that may be provided upon the examination result screen, for example, and thus select the dates and times to make the make the appointments from among the dates and times when the appointments for examinations can be made displayed on this screen, and then send the selected information to the appointment-making system 10.

Upon receiving the dates and times when the appointments for examinations can be made that are selected from the physician's terminal 20, by executing the appointment date and time setting means, the appointment-making system 10 sets them within the time frame in which appointments can be made and stores them in the examination appointment situation database 110 (Step 10). Thus, the dates and times for the examination appointments are set. Accordingly, the physician can easily make appointments for examinations even within the limited time of a consultation.

(2) Making Appointments for Post-Examination Consultations

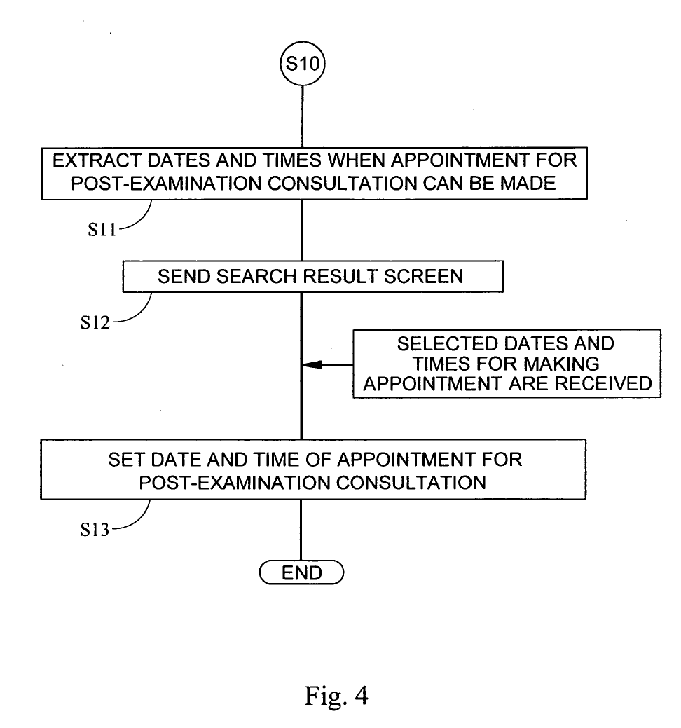

With this appointment-making system 10, it is possible to not only make appointments for examinations but also, it is possible to make appointments for post-examination consultations even further on. To wit, as shown in the flowchart of FIG. 4, if the set dates and times for the examination appointments are stored in the examination appointment situation database 110 by means of Step 10 above, next, by executing the consultation appointment date and time search means, based on the date when the results for the examination in question will be obtained as computed in Step 8, the system accesses the consultation appointment situation database 130 and makes a query on the appointment situation for consultations stored in this database 130, and thus extracts one or more dates and times at which a post-examination consultation can be made (Step 11). Note that if two or more examinations were specified and the dates when the results of these examinations will be obtained are different, then the latter of the dates when the examinations will be obtained will be used as the basis. For example, if the dates when the results of examination A and examination B will be obtained are July 20 and July 25, respectively, then July 25 which is the date when the results of examination B will be obtained will be used as the basis for extracting dates and times when the appointment for a post-examination consultation can be made.

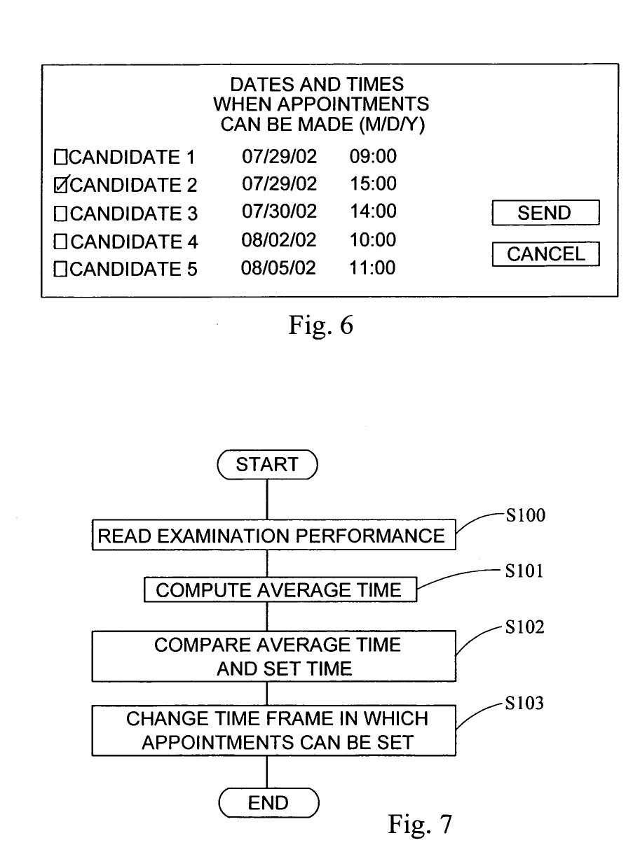

Once the extraction of the dates and times when the appointment for the post-examination consultation can be made is performed, next, by executing the information sending means, the system sends a search result screen containing the dates and times when the appointment for the post-examination consultation can be made as extracted in Steps 11 to the physician's terminal (Step 12). Thereby, a search result screen such as that illustrated in FIG. 6, for example, is displayed upon the display of the physician's terminal 20.

In the same manner as when making appointments for examinations, the physician may ask the patient for an opinion and add this to the physician's own judgment and then check one of the checkboxes that may be provided upon the examination result screen, for example, and thus select the dates and times to make the make the appointments from among the dates and times when the appointment for the post-examination consultation can be made displayed on this screen, and then send the selected information to the appointment-making system 10.

Upon receiving the date and time when the appointment for the post-examination consultation can be made that is selected from the physician's terminal 20, by executing the appointment date and time setting means, the appointment-making system 10 sets it within the time frame in which appointments can be made and stores it in the consultation appointment situation database 130 (Step 13). Thus, the date and time for the appointment for the post-examination consultation are set.

Accordingly, with this appointment-making system 10, the patient can make an appointment for a post-examination consultation together with the appointments for examinations, so there is no need to make an appointment for a consultation again after the examinations.

(3) Making Appointments for Regularly-Scheduled Examinations, etc.

Appointments for regularly-scheduled examinations and the like can be made as follows. To wit, in this case, the search condition input screen that is sent in Step 1 above may include: a field used to select whether the examination is a regularly-scheduled examination or not, the time period between examinations, the number of times the examination is to be repeated and other input items. For example, if the field to indicate a regularly-scheduled examination is selected, the time period between examinations is specified to be one month and the number of times to repeat the examination is specified as 12 times, then upon receiving these search conditions, the appointment-making system 10 first executes the processing of Steps 2 to 8 above for the examination to be performed first. Next, the system again executes the processing of Steps 2 to 8 above taking a date roughly one month later than the first date and time when an appointment for an examination can be made as extracted in Step 7 above (including several days before or after the response date) as the search condition. Thereafter, the system repeats the same process an additional 10 times. As a result, the dates and times when 12 appointments for regularly-scheduled examinations at one-month intervals can be made are extracted and also, the dates when the results of each instance of the examination will be obtained will be computed. Moreover, in Step 9 above, the system sends the dates and times when the appointment for the examinations can be made as extracted for each instance of the examination to the physician's terminal 20. Thereby, in the same manner as above, a search result screen containing dates and times when appointments for examinations can be made as extracted for each instance of the examination is displayed upon the display of the physician's terminal 20 in a form such that the physician or patient can select among several candidates.

Upon receiving the dates and times when the appointments for examinations can be made for each instance of the examination that are selected from the physician's terminal 20, the appointment-making system 10 sets them within the time frame in which appointments can be made that is prepared in the examination appointment situation database 110 and stores them in the examination appointment situation database 110. Thus, by performing the processes in Steps 11 through 13 above, the dates and times for appointments for each instance of the examination can be set.

Thus, appointments for regularly-scheduled examinations and appointments for post-examination consultations can be easily made with such an appointment-making system 10.

Here follows a description of the second embodiment of the present invention.

(4) Updating Anticipated Examination Times

The second embodiment is characterized in that the amount of time required for each examination is set separately for each patient undergoing the examination, technician or physician performing the examination, respectively. Moreover, in the operation, the examination times are updated at regular intervals.

As described above, the appointment situation for examinations is stored in the examination appointment situation database 110. When scheduling examination appointments in the first place, it is important to accurately determine and anticipate the examination times for each examination item. However, it is extremely difficult to predict the amount of time required for each examination in all cases. For example, even for the same examination, the examination times will vary depending on the age, sex and bodily condition of the patient undergoing the examination. The bodily condition may be any physical conditions of the patient, such as “impossible to walk,” “need a hearing aid,” etc. In addition, the examination times will also vary depending on the examination technician and physician administering the examination. Furthermore, in addition to these “static” factors, it is also necessary to consider “dynamic” factors in anticipating the examination times. These “dynamic” factors include, for example, the proficiency and technical skills of the technician or physician conducting the examination. The examination times are expected to become shorter as the proficiency of the technician or physician improves. In addition, the examination times will also vary with changes to the examination process and as the examination equipment ages.

To solve this problem, an examination technician or physician or other personnel, every time he or she conducts an examination, using one of the terminals 20 and 30 installed in various locations, records his or her examination performance for the particular examination, or namely the actual time he or she needed to conduct that examination. In FIG. 2, the performance data inputted from the terminals 20 and 30 is sent through the network 40 to the appointment-making system 10, where it is recorded in the examination performance database 140. In addition, when recording the examination times, the examination situation is also recorded at the same time. This examination situation includes the age, sex and bodily condition of the patient who underwent the examination, as well as the name of the examination technician or physician who administered the examination. When the performance is recorded in this manner, the anticipated examination time is regularly updated based on the performance recorded in the past.

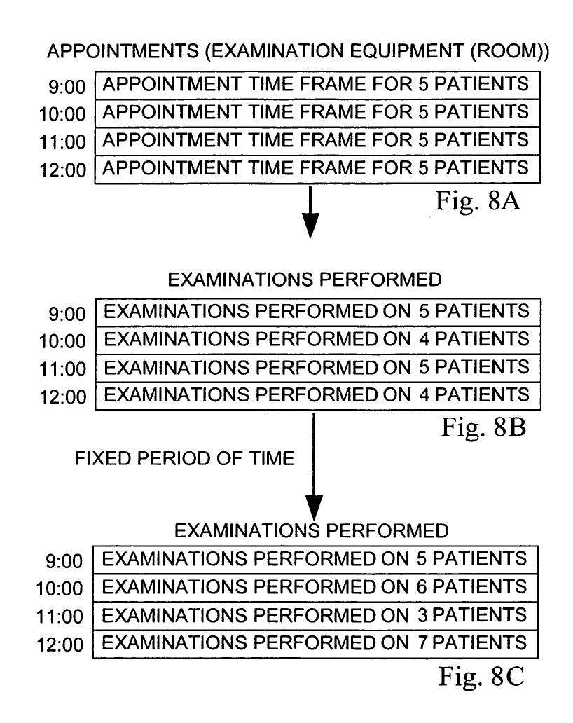

Specifically, the anticipated examination times are updated according to the flowchart shown in FIG. 7. As described above, the examination performance database 140 stores the time required for an examination for each instance of the examination. This time required for the examination is recorded together with the age, sex and bodily condition of the patient who underwent the examination, as well as the name of the examination technician or physician who administered the examination. The performance data thus recorded is read by the main controller 11 at regular intervals, for example, once every week (Step 100). Then, for each individual examination, the main controller 11 then calculates the average of the examination times recorded over a fixed period in the past, for example, three months (Step 101). This average time is calculated for each of the “static” and “dynamic” factors described above. As a result, for a single examination, the average examination time is found for each of the individual technicians or physicians, and for the age, sex and bodily condition of the patient who underwent the examination, respectively. For example, for a specific examination, the examination time required for technician A to perform an examination on a 50-59 year old male patient over the past three months, the examination time required for a female patient of the same age, the examination time required for a 40-49 year old male patient and the like, respectively, are each computed.

The main controller 11 then compares the average examination times thus computed against the examination times currently set and used as the examination times (the set times) (Step 102). If there is a large difference between the two, the main controller sets the average time thus computed as the new set times.

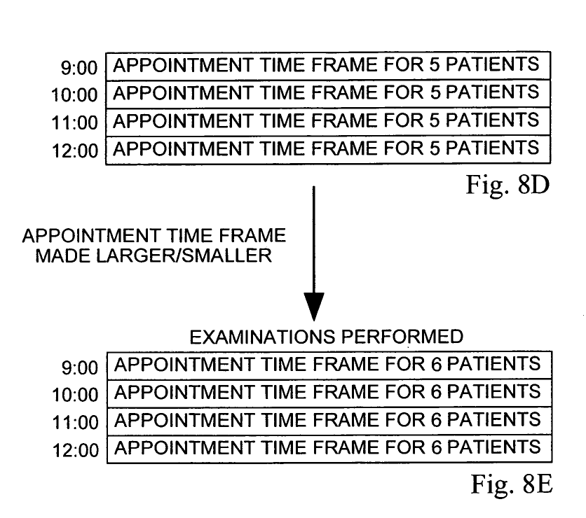

Here follows a more detailed description of the updating of these average examination times made using FIG. 8. We shall assume that the average examination time for a particular examination room is at issue which is provided with examination equipment that performs a certain specific examination. We also assume that upon taking the past average, the results indicate that an average of five persons can be examined per hour in that room. Thus, a time frame for patients is set up so that five persons can be examined in a one-hour time frame (FIG. 8(A)). Suppose that on the first day when the number of patients was so set for each time frame, patients were actually examined as shown in FIG. 8(B), which shows the number of patients actually examined within each time frame. To wit, where initially five patients were assumed to undergo the examination per hour, five patients were actually examined in the time frames from 9:00 to 10:00 and from 11:00 to 12:00, respectively, but only four patients were examined in the timeframes from 10:00 to 11:00 and from 12:00 to 1:00.

However, the “dynamic” factors described above act over time so that when an average is taken of the examination performance three months later, the result changed as shown in FIG. 8(C). To wit, an average of six persons were found to be examined in the time frame from 10:00 to 11:00, and an average of seven persons were examined in the time frame from 12:00 to 1:00. The fact that there is dispersion in the number of people among the time frames as an average value may be related to the “static” factors described above. However, although the “static” factors affect the average, one can see that it is possible to examination seven people per hour when the efficiency of examination is greatest. Moreover, if one takes the average number of people per one-hour time frame in this examination room from FIG. 8(C), this is 5.25 people. From this, one can judge that at the initial setting of five people per hour, the examination equipment and examination technicians are not working under conditions where they can work efficiently enough. Thus, based on these results, the initial setting of five people per hour as shown in FIG. 8(D) may be changed to six people per hour as shown in FIG. 8(E). Thus, at the updated settings for examination appointments, this examination room will be able to accept six people per one-hour time frame.

Note that the description above presents the case of increasing the number of patients undergoing examinations per hour, but the “dynamic” factors may also act in the negative direction. For example, the examination efficiency may decrease as examination equipment ages. In the case that the average number of patients undergoing examination per one-hour time frame drops below the setting, then the set number of patients undergoing examination per hour will be reduced correspondingly.

(5) Making Examination Appointments in Consideration of Examination Performance

Here follows a description of making appointments for examinations in the second embodiment made with reference to FIG. 3. However, in this description of the second embodiment, only those processes that are different from the first embodiment, which has already been discussed above, will be described in order to avoid redundant explanation.

In the same manner as in the first embodiment, the physician makes an appointment from the terminal 20. At this time, in addition to the input items described in the first embodiment, the physician also inputs the sex and age of the patient undergoing the examination (Step 1). After performing Steps 2-6 of FIG. 3, the appointment-making system 10 accesses the examination appointment situation database 110 and queries the appointment situation while extracting one or more dates and times at which appointments may be made for each of the required examinations (Step 7). At this time, the examination performance database 140 is also accessed simultaneously to query the examination times set for the technician and physician to perform the examinations. Then, the appointment-making system 10 reads the amount of time that the technician or physician to perform the examinations is anticipated to require to perform the examinations, and uses the anticipated times thus read out to extract the dates and times at which appointments may be made. Moreover, the dates and times of the appointments selected via Steps 8 and 9 are stored in the examination appointment situation database 110 together with the amount of time required for the examination, namely the examination times stored in the examination performance database 140.

(6) Updating the Examination Appointment Situation

When the number of patients that can undergo examinations per one-hour time frame is updated as described above (e.g., when the number of patients per hour is increased from five to six), the updated number of patients is used thereafter when accepting subsequent appointments.

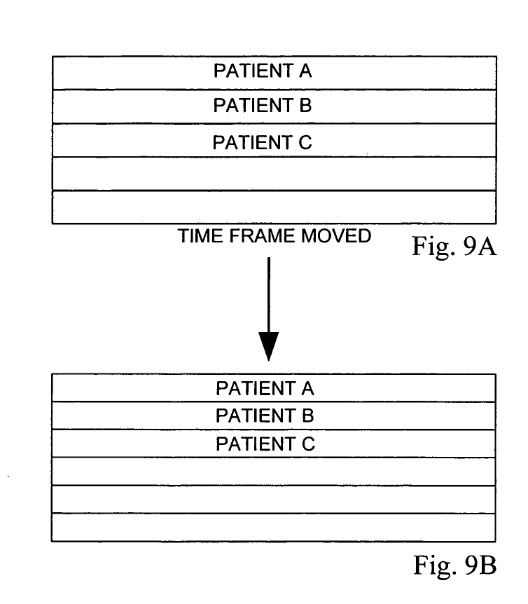

In addition, appointments are typically made up to several weeks in the future. To wit, at the point in time when the update is made, most appointments will have been already filled up until several weeks in the future. It is difficult (but not impossible) to change the times of appointments for patients that have already made appointments. Thus, the appointments for patients that have already made appointments are to be given priority and remain unchanged. However, after the update, the situation will change for patients who are making new appointments. To wit, in Step 7 of FIG. 3 described above, one or more dates and times when appointments can be made are extracted, but the fact that the number of patients permitted to undergo examinations per hour is updated will affect this extraction process. For example, assume that the setting for the number of patients per one-hour time frame is five as shown in FIG. 9(A) and the three patients A, B and C have currently made appointments within a certain time frame on a certain future date. In FIG. 9(A), appointments for two additional patients can be made within that specific time frame. However, when the setting of five people is updated to a setting of six, for example, it is now possible to make appointments for three additional patients for the time frame as shown in FIG. 9(B).

As described above, with the present invention, appointments for examinations and the like can be easily made by a physician during consultations. In addition, the physician is able to determine the dates when examination results will be obtained, so an appropriate consultation schedule can be set up. Moreover, not only will it simply improve the efficiency of the work of making appointment but it can achieve enhanced efficiency in the work of the examinations for which appointments are made.

It will be appreciated by those skilled in this art that various modifications and variations can be made to the above-embodiments without departing from the spirit and scope of the invention. Other embodiments of the invention will be apparent to those skilled in this art from consideration of the specification and practice of the invention disclosed therein. It is intended that the specification and examples be considered exemplary only, with a true scope and spirit of the invention being indicated by the following claims.

1. A method of making a medical examination appointment, comprising the steps of:
storing performance data for each instance of examination;
updating, based on the stored performance data, a number of appointments acceptable for each time frame;
receiving an appointment request designating at least one kind of examination;
determining one or more time frames available to accept the appointment request, wherein determining one or more time frames comprises (a) determining if a physician needs to attend the examination, (b) determining if the examinations can be conducted on the same day if more than one kind of examination is designated, and (c) determining priority among the examinations if more than one kind of examination is designated; and
presenting the available one or more time frames for selection.

2. A method according to claim 1, wherein the performance data comprises a time actually spent for an examination, the identity of a person who conducted the examination, and attributes of a patient who underwent the examination.

3. A method according to claim 2, wherein the attributes comprise any of the age, sex and bodily condition of the patient.

4. A method according to claim 1, wherein updating a number of appointments acceptable for each time frame comprises averaging a number of patients actually examined over a period of time for each time frame.

5. A method according to claim 1, wherein updating a number of appointments acceptable for each time frame comprises leaving intact appointments already made before the update.

6. A method according to claim 1, wherein updating a number of appointments acceptable for each time frame comprises increasing or decreasing a number of appointments acceptable for a time frame.

7. A method according to claim 1, wherein presenting the available one or more time frames for selection comprises presenting, for each of the available one or more time frames, a date when results of examination become available.

8. A method according to claim 1, wherein determining one or more time frames available to accept the appointment request comprises selecting a set of available time frames whose time intervals between the time frames are shortest if more than one time frame is determined necessary to fill the appointment and if more than one set of time frames are available for the appointment.

9. A method according to claim 1, further comprising presenting time slots for selection which are available for post-examination consultation with a physician, wherein time slots are determined based on dates when examination results become available.

10. A medical examination appointment making system, comprising:
a memory that stores performance data for each instance of examination;
a performance manager that updates, based on the stored performance data, a number of appointments acceptable for each time frame;
an input unit that receives an appointment request designating at least one kind of examination;
a timeframe finder that determines one or more time frames available to accept the appointment request, wherein when determining one or more time frames, the timeframe finder determines (a) if a physician needs to attend the examination, (b) if the examinations can be conducted on the same day if more than one kind of examination is designated, and (c) priority among the examinations if more than one kind of examination is designated; and
an output unit that presents the available one or more time frames for selection.

11. A system according to claim 10, wherein the performance data comprises a time actually spent for an examination, the identity of a person who conducted the examination, and attributes of a patient who underwent the examination.

12. A system according to claim 11, wherein the attributes comprise any of the age, sex and bodily condition of the patient.

13. A system according to claim 10, wherein the performance manager, when updating a number of appointments acceptable for each time frame, averages a number of patients actually examined over a period of time for each time frame.

14. A system according to claim 10, wherein the performance manager, when updating a number of appointments acceptable for each time frame, leaves intact appointments already made before the update.

15. A system according to claim 10, wherein the performance manager, when updating a number of appointments acceptable for each time frame, increases or decreases a number of appointments acceptable for a time frame.

16. A system according to claim 10, wherein the output unit, in addition to presenting the available one or more time frames for selection, presents, for each of the available one or more time frames, a date when results of examination become available.

17. A system according to claim 10, wherein the timeframe finder selects a set of available time frames whose time intervals between the time frames are shortest if more than one time frame is determined necessary to fill the appointment and if more than one set of time frames are available for the appointment.

18. A system according to claim 10, wherein the output unit further presents time slots for selection which are available for post-examination consultation with a physician, wherein time slots are determined based on dates when examination results become available.
